(19) United States
(12) Patent Application Publication
(10) Pub. No.: US 2015/0353951 A1
(43) Pub. Date: Dec. 10, 2015

(54) SYNTHETIC GLYPHOSATE-RESISTANT GENE AND USE THEREOF

(71) Applicant: ORIGIN AGRITECH LIMITED, Beijing (CN)

(72) Inventors: Gengchen Han, Beijing (CN); Fukun Jiang, Beijing (CN); Dezhi Deng, Beijing (CN); Xuefeng Li, Beiing (CN); Zhe Song, Beijing (CN)

(73) Assignee: ORIGIN AGRITECH LIMITED, Beijing (CN)

(21) Appl. No.: 14/391,948

(22) PCT Filed: Apr. 12, 2013
(86) PCT No.: PCT/CN2013/000428

§ 371 Date: Oct. 10, 2014

(30) Foreign Application Priority Data
Apr. 12, 2012 (CN) 201210107071.2
Apr. 12, 2012 (CN) 201210107195.0
Apr. 12, 2012 (CN) 201210107400.3
Apr. 12, 2012 (CN) 201210107425.3

(51) Int. Cl. C12N 15/82; C07K 14/415

(52) U.S. Cl. CPC C12N 15/8275; C07K 14/415

(57) ABSTRACT

Disclosed in the present invention is a synthetic glyphosate-resistant gene and the use thereof. the gene provided in the present invention in one of following (a)-(c): (a) a DNA molecule having a nucleotide sequence shown as sequence 2 the sequence listing; (b) a DNA molecule having nucleotide sequence shown as positions 1-1335 of sequence 2 in the sequence listing; (c) a DNA molecule having a nucleotide sequence having an identity of at least 98% with the sequence 2 or the position 1-1335 of the sequence 2 in the sequence listing and encoding a protein shown as sequence 9. Experiment demonstrates that the transgenic maize with the synthetic glyphosate-resistant gene provided by the present invention has significantly increased G2-aroA protein expression and significantly improved tolerance to glyphosate compared with the transgenic maize with the prokaryote glyphosate-resistant gene G2-aroA.

### CROSS-REFERENCE TO RELATED APPLICATIONS

The present application is a U.S. National Stage of International Application No. PCT/CN2013/000428, filed Apr. 12, 2013, which claims the benefit of Chinese Patent Applications 201210107071.2, 201210107195.0, 201210107425.3, and 201210107400.3, each filed on Apr. 12, 2012, the contents of which are incorporated herein by reference in their entireties.

### SEQUENCE LISTING

The instant application contains a Sequence Listing which has been submitted electronically in ASCII format and is hereby incorporated by reference in its entirety. Said ASCII copy, created on Jan. 26, 2015, is named PCTCN2013000428-Updated Sequence Listing.txt and is 34,380 bytes in size.

### TECHNICAL FIELD

The present invention relates to an synthetic glyphosate-resistant gene and the use thereof, especially to a glyphosate-resistant gene design and synthesized according to maize codon usage bias, and the use thereof.

### BACKGROUND OF THE INVENTION

At present, most of exogenous genes, such as Bt, EPSPS and the like used in plant transgenic breeding are from prokaryote. Since the characteristics of genes of prokaryote, such as 1) higher content of AT, greater than 60%, makes mRNAs expressed in plant easily degraded; 2) existing of intron splice sites, transcription terminators sequences that are similar to those in eukaryotic genes, causes incompletely transcription and abnormal mRNA splicing; 3) the greater difference between codons components of prokaryote and that of plants, makes a lower protein translation efficacy; 4) there are notable differences between gene structures of prokaryote and that of eukaryotic organisms, such as plants, for example, the gene structures of prokaryote do not contain 5′-UTR sequences and 3′-terminus polyA tails sequences as contained in genes of eukaryotic organisms, typically causing the gene of prokaryote expressed in a lower level in plants. For example, the expression level of wild type insecticidal protein genes derived from Bacillus thuringiensis in plants is very low, the expressed toxic proteins comprise only 0.001% of the total proteins, and otherwise it almost can not be detected. Perlak in Monsanto Company, U.S.A (Perlak F J, Fuchs R L, Dean D A, et al. Modification of the coding sequence enhances plant expressing of insect control protein genes. Proc Natl Acad Sci USA, 1991, 88:3324-3328) and Iannacone et al., (Iannacoe R, Grieco P D, Cellini F. Specific sequence modification of a cry3B endotoxin gene result in high levels of expression and insect resistance. Plant Mol Biol, 1997, 34: 485-496) modified the insecticidal proteins genes and cryIII genes, respectively, provided that the toxic proteins amino acids sequences are not changed. They used codons that are preferable for plants, increased the content of GC, and removed polyA and ATTTA sequences riching in AT, and other unstable element sequences existed in original sequences. Therefore, the expression amount of toxic proteins in the transgenic plants increased by 30-100 folds, comprising 0.02-1% of soluble proteins, and achieved a significant insect-resistance effects.

Weeds are a great risk to field crops production, since appearance of weeds prevention technology in 1942, chemical herbicide processed quickly. Glyphosate herbicide is a broad spectrum, non-selective herbicide. It inhibits the activity of EPSPS (5-enolpyruvyl-shikimate-3-phosphate synthase, which is an important enzyme for aromatic amino acids synthesis pathway in plants), blocks biosynthesis of shikimic acid pathway in plants, notably inhibits cell division, and has notable inhibition effects on both annual and perennial weeds. Because glyphosate is easily decomposed by microorganisms, no residual poison exists in soil, no toxic effect to animals, it is used widely since successfully developed by Roundup in 1976.

However, since Gramineae crops such as maize are sensitive to glyphosate, its usage is limited. Therefore, if glyphosate-resistant gene can be transferred into maize, not only the usage range of glyphosate can be broadened, but also the production cost can be decrease. The maize can be prevented from damage by herbicide, and finally to achieve an increased production aim. Although the glyphosate-resistant gene G2-aroA had been discovered in 2004, and can be expressed in host Pseudomonas fluorescens G2 and Escherichia coli effectively to show a high glyphosate-resistance trait (Yichen Sun, Min Lin and Yiping Wang. Novel AroA with high tolerance to Glyposate, encoded by a gene of Pseudomonas putida 4G-1 isolated from an extremely polluted environment in China Applied and environmental microbiology. 2005, 71(8):4771-4776), since expression amount in plants, especially in major food crops is low, it is not used. Therefore, it is a great need to research its use in plants, such as codon optimization, so as to facilitate its use in agricultural production.

### SUMMARY OF THE INVENTION

An aim of present invention is to provide a DNA molecule and the use thereof, the DNA molecule is a glyphosate-resistant gene.

the DNA molecule provided by present invention is designated as mG2-aroA, its nucleotide sequence is the SEQ ID NO. 2 in the sequence listing.

As needed, nucleotide sequence of the DNA molecule can also be those shown as positions 1-1335 of the SEQ ID NO. 2 in the sequence listing.

The nucleotide sequence of the DNA molecule can also be those having an identity of at least 98% with SEQ ID NO. 2 or positions 1-1335 of SEQ ID NO. 2 in the sequence listing, and encoding the protein shown in SEQ ID NO. 9 (designated as G2-aroA protein).

The expression cassette, recombinant vector, recombinant host bacteria, recombinant cell line or transgenic plant containing the DNA molecule also fall into the protection scope of present invention.

The expression cassette can specifically contain the following 1)-3) elements: 1) a promoter; 2) the DNA molecule starting transcription by the promoter; 3) a transcription terminator sequence.

As needed, the recombinant vector can be both a recombinant cloning vector and a recombinant expression vector.

Available plant expression vectors at present can be used to construct the recombinant expression vector expressing the glyphosate-resistant gene. The plant expression vectors include a binary Agrobacterium tumefaciens vector and vectors that can be used in plant Microprojectile such as pROKII, pBin438, pCAMBIA1302, pCAMBIA2301, pCAMBIA1301, pCAMBIA1300, pBI121, pCAMBIA1391-Xa or pCAMBIA1391-Xb (CAMBIA Company). In an example of present invention, the recombinant vector is a pS3300-UMG2.

In an example of present invention, the recombinant host bacteria are Agrobacterium tumefaciens carrying the DNA molecule, such as LBA4404.

The recombinant cell line can be a eukaryotic cell, and a prokaryotic cell, such as a plant cell line.

The transgenic plant (such as maize) includes seeds, callus, a whole plant and a cell.

In the expression cassette or the recombinant expression vector, the promoter that suitable for present invention includes but not limits to a constitutive promoter, a tissue, organ and development specific promoter, and a inducible promoter, such as constitutive promoter 35S of the cauliflower mosaic virus; tomato protease inhibitor II promoter (PIN2) or LAP promoter (both can be induced by methyl jasmonate); heat shock promoter; tetracycline inducible promoter; seeds specific promoter, such as millet seeds specific promoter pF128, seeds storage proteins specific promoter, for example, promoters of Phaseolin, napin, oleosin and bean beta conglycin.

In an example of present invention, the promoter in the expression cassette or the recombinant expression vector that start the DNA molecule (mG2-aroA gene) is the Ubi promoter, wherein the nucleotide sequences is the SEQ ID NO. 5 in the sequence listing, or a sequence having an identity of at least 80% with SEQ ID NO. 5, and having promoter function.

In the expression cassette or the recombinant expression vector, transcription terminators suitable for the present invention includes but not limits to Agrobacterium tumefaciens nopaline synthase terminator (NOS terminator), cauliflower mosaic virus CaMV 35S terminator, tml terminator, pea rbcS E9 terminator and nopaline and octopine synthase terminators.

In an example of present invention, the transcription terminator sequences in the expression cassette or the recombinant expression vector is specifically a T-NOS double terminator sequence, such as shown in positions 216-491 of SEQ ID NO. 8 in the sequence listing, or sequences having an identity of at least 80% with positions 216-491 of SEQ ID NO. 8 and possessing transcription termination function.

In an example of present invention, the expression cassette or the recombinant expression vector further include OMK sequence. The OMK sequence consists of Ω sequence and Kozak sequence that connected in succession, and its sequence is specifically SEQ ID NO. 6 in the sequence listing, or a sequence having an identity of at least 80% with SEQ ID NO. 6 and possessing enhancer function.

The Ω sequence and Kozak sequence derived from tobacco mosaic virus, are enhancers responsible for enhancing expression of the glyphosate-resistant gene.

In an example of present invention, the expression cassette or the recombinant expression vector further include a chloroplast transit peptide (CTP) sequence, and its sequence is specifically SEQ ID NO. 7 in the sequence listing, or a sequence having an identity of at least 80% with SEQ ID NO. 7 and possessing signal peptide function.

The chloroplast transit peptide (CTP) sequences derived from maize is a signal peptide sequence, which can transport proteins (G2-aroA protein) expressed by the glyphosate-resistant gene to chloroplast.

In an example of present invention, the expression cassette consists of the Ubi promoter, the OMK sequence, the chloroplast transit peptide sequence, the glyphosate-resistant gene and the transcription terminator sequences connected in succession, designated as Ubi-OMK-CTP-mG2aroA-PolyA-T-NOS; its sequence is specifically shown as SEQ ID NO. 10 in the sequence listing.

In an example of present invention, the sequence of the recombinant expression vector (designated as pS3300-UMG2) is the SEQ ID NO. 11 in the sequence listing.

The resulted RNA transcripted from the DNA molecule also falls into the protection scope of present invention.

The use of the DNA molecule (mG2-aroA gene), or the expression cassette, or the recombinant vector in breeding glyphosate resistant transgenic maize also falls into the protection scope of present invention. The transgenic maize includes seeds, callus, whole plants and cells.

The use of the DNA molecule (mG2-aroA gene) in increasing maize G2-aroA protein expression amount also falls into the protection scope of present invention. The G2-aroA protein is that shown in SEQ ID NO. 9 of the sequence listing.

Wherein, SEQ ID NO. 2 consists of 1338 nucleotides, at the end thereof there are two termination codons. The positions 1-1335 of SEQ ID NO. 2 are a encoding sequence, encoding G2-aroA protein shown in SEQ ID NO. 9 of the sequence listing. The SEQ ID NO. 5 consists of 2010 nucleotides. The SEQ ID NO. 6 consists of 67 nucleotides. The SEQ ID NO. 7 consists of 141 nucleotides. The SEQ ID NO. 8 consists of 491 nucleotides. The SEQ ID NO. 9 consists of 444 amino acids. The SEQ ID NO. 10 consists of 4058 nucleotides, wherein positions 1-2010 are Ubi promoter sequence, positions 2022-2088 are OMK sequence, positions 2089-2229 are chloroplast transit peptides (CTP) sequence, positions 2230-3567 are mG2-aroA sequence, positions 3568-4058 are transcription terminator sequence. The SEQ ID NO. 11 consists of 10488 nucleotides, wherein positions 151-2165 are Ubi promoter sequence, positions 2177-2243 are OMK sequence, positions 2244-2384 are a CTP sequence, positions 2385-3722 are mG2-aroA sequence, positions 3723-4213 are transcription terminator sequence.

Another aim of present invention is to provide a method for breeding glyphosate resistant transgenic maize.

The method provided by present invention for breeding glyphosate resistant transgenic maize specifically includes the following steps: introducing the DNA molecule (mG2-aroA gene) into a target maize, so as to obtain a transgenic maize expressing the DNA molecule (mG2-aroA gene); the transgenic maize has an increased resistance to glyphosate as compared to the target maize.

Wherein, the DNA molecule (mG2-aroA gene) can be modified as follows, and then introduced into host, so as to obtain a better expression effect:

1) In order to expression of gene effectively, the modification and optimization are made as actual need; for example, according to receptor plant biased codons, the condons can be changed while maintaining the amino acids sequence of glyphosate resistant proteins unchanged, so as to meet the plant's bias; at the same time, the content of GC can be maintained to achieve high level expression of genes introduced into plant, wherein content of GC can be 35%, greater than 45%, greater than 50% or greater than about 60%;

2) Modifying the genes sequence neighboring the start methionine so as to effectively start translation; for example, to modify using sequences known effective in plants;

3) Connecting various of promoters expressed by plants to facilitate its expression in plants; the promoter may include constitutive, inducible, temporal regulation, developmental regulation, chemical regulation, tissues preferable and tissues specific promoters; the selection of promoters my vary according to expression time and space as well as target species; for example specific expression promoter of tissues or organs are determined as needed of receptor development time; although it is proved that many promoters derived from dicotyledon can function in monocotyledon, vice versa. Ideally, however, the promoters of dicotyledon are chosen for expression in dicotyledon, and the promoters derived from monocotyledon are used for expression in monocotyledon;

4) Connecting to suitable transcription terminators can also increase expression efficacy of genes of present invention; for example tml derived from CaMV, E9 derived from rbcS; any terminators known function in plants and available can be connected to genes of present invention;

5) Introducing enhancer sequences, such as intron sequences (for example those derived from Adhl and bronzel) and virus leader sequences (for example those derived from TMV, MCMV and AMV).

In above method, introducing the DNA molecule into target maize is achieved by introducing the recombinant expression vector into the target maize.

The recombinant expression vector can be introduced into plant cells by using Ti plasmids, plant virus vector, direct DNA transformation, microinjection, electroporation and other common technology methods.

The transgenic maize obtained by using the method provided by present invention for breeding glyphosate resistant transgenic maize also falls into the protection scope of present invention. The transgenic maize includes seeds, callus, whole plants and cells.

In an example of present invention, the maize is specifically maize cultivar, zong31.

Regarding the issue that prokaryote glyphosate-resistant gene G2-aroA (SEQ ID NO. 1) is expressed in plant with a low efficiency, in the present invention, the gene G2-aroA is optimized by the maize biased codons, and structures that influence stability of RNA (such as polyA, repeat sequences, AT and GC tandem repeat, RNA secondary structures, ribosome bind site, and the like) are removed, and the content of GC is increased, as long as the initial amino acids sequence is maintained unchanged, so as to make the gene G2-aroA express in maize effectively and stably.

### DESCRIPTION OF DRAWINGS

FIG. 1 represents the plasmid map of the vector pUC19-UG2.

FIG. 2 represents the plasmid map of the vector pCAMBIA3300.

FIG. 3 represents the plasmid map of the vector pS3300.

FIG. 4 represents the plasmid map of the recombinant expression vector pS3300-UG2.

FIG. 5 represents the plasmid map of the recombinant expression vector pS3300-UMG2.

FIG. 6 represents the plasmid map of the recombinant vector pS3300-UG0

FIG. 7 represents the PCR identification picture of T₆ generation G2-aroA transgenic maize. Wherein, lane 1 represents the positive control; lane 2 represents the DNA Marker (D2000); lane 3 represents the blank control group; lane 4 represents the non-transgenic maize plant (negative control 2); lanes 5-15 represents the 11 T₆ generation transgenic maize plants transformed with G2-aroA gene.

FIG. 8 represents the PCR identification picture of the T₆ generation mG2-aroA transgenic maize. Wherein, lane 1 represents the positive control; lane 2 represents the DNA Marker (D2000); lane 3 represents the blank control group; lane 4 represents the non-transgenic maize plants (negative control 2); lanes 5-24 represent the 20 T₆ generation transgenic maize plants transformed with G2-aroA gene.

FIG. 9 represents filed identification map of glyphosate resistance of the T₆ generation G2-aroA transgenic maize plants and T₆ generation mG2-aroA transgenic maize plants. Wherein, A represents the mG2-aroA transgenic maize plants, blank vector transgenic plants and non-transgenic plants; B represents the mG2-aroA transgenic maize plants and G2-aroA transgenic maize plants. In A and B, the maize line shown as 1 represents the mG2-aroA transgenic maize plants; the maize line shown as 2 represents the G2-aroA transgenic maize plants; the maize line shown as 3 represents the blank vector transgenic maize plants; the maize line shown as 4 represents the non-transgenic maize plants.

FIG. 10 represents a standard curve of G2-aroA protein concentration detected by double-antibody sandwich ELISA.

FIG. 11 represents a SDS-PAGE electrophoresis identification picture of the purified G2-aroA protein. Wherein, lane 1 represents the proteins Marker, from top to bottom are 72KD, 45KD, 32KD, 14.4KD, respectively; lanes 2-4 all represent purified G2-aroA protein after expression of the prokaryotic recombinant expression vector pET-28a-G2-aroA, and the loading amounts are 5 μl, 10 μl and 15 μl, respectively.

FIG. 12 represents purified monoclonal antibodies concentration detected by SDS-PAGE. Wherein, lane 1 represents the protein Marker; lane 2 represents the purified monoclonal antibodies, the larger target band represents the heavy chain, and the smaller target band represents the light chain.

### EXAMPLES

The experimental methods used in the following Examples are common methods, unless specified otherwise.

The materials, agents used in the following Examples are all commercially available, unless specified otherwise.

### Example 1

### Preparation of Codons Optimized Glyphosate-Resistant Gene

This Example is on the basis of amino acids sequence (nucleotide sequences shown as SEQ ID NO. 1 of the sequence listing) of G2-aroA gene (Chinese Patent application No. 03826892.2, grant publication No. CN 100429311 C; U.S. Pat. No. 913,651, U.S. Pat. No. 7,238,508). On the premise that the amino acids sequence are not changed, the G2-aroA gene is firstly artificially optimized using maize biased codons. It is better to avoid using of rare codons of maize, and the usage frequency of condons is adjusted (Table 1). Based on above, sequences AT-rich that typically cause plant genes transcript unstable are removed from DNA sequences, as well as the hairpin structure removed, to obtain new nucleotide sequence shown as SEQ ID NO. 2 in the sequence listing. The identity between SEQ ID NO. 2 and G2-aroA gene (SEQ ID NO. 1) is only 84%, and the content of G+C is decreased from initial 64.83% to 62.07%. The gene shown in SEQ ID NO. 2 is the codons optimized glyphosate-resistant gene, designated as mG2-aroA. The usage frequency of codons in G2-aroA gene and mG2-aroA gene can be see in Table 1. In order to facilitate cloning, we introduced a BamH I restriction site at 5′ terminus of SEQ ID NO. 2, and a Kpn I restriction site at 3′ terminus, and the resulted sequence is shown as SEQ ID NO. 3 of the sequence listing. The positions 7-1344 of SEQ ID NO. 3 is the SEQ ID NO. 2. The SEQ ID NO. 1, positions 1-1335 of SEQ ID NO. 2, and positions 7-1341 of SEQ ID NO. 3 are all encoding sequences. They all encode the protein shown in SEQ ID NO. 9 of the sequence listing, and the protein is designated as G2-aroA protein.

TABLE 1
Standard for optimized codons in maize

| | | usage frequency of codons | |
|---|---|---|---|
| | | G2-aroA gene % | mG2-aroA gene % |
| | | (number of | (number of |
| amino acids | codons | amino acids) | amino acids) |
| Lys | AAA | 42.9 (6) | 7.1 (1) |
| | AAG | 57.1 (8) | 92.9 (13) |
| Asn | AAC | 100 (10) | 80 (8) |
| | AAT | 0 (0) | 20 (2) |
| Thr | ACA | 2.6 (1) | 10.5 (4) |
| | ACC | 78.9 (30) | 47.4 (18) |
| | ACG | 13.2 (5) | 28.9 (11) |
| | ACT | 5.3 (2) | 13.2 (5) |
| Arg | AGA | 0 (0) | 11.1 (2) |
| | AGG | 0 (0) | 27.8 (5) |
| | CGA | 0 (0) | 0 (0) |
| | CGC | 55.6 (10) | 38.9 (7) |
| | CGG | 5.6 (1) | 11.1 (2) |
| | CGT | 38.9 (7) | 11.1 (2) |
| Ser | AGC | 38.1 (8) | 28.6 (6) |
| | AGT | 4.8 (1) | 4.8 (1) |
| | TCA | 0 (0) | 9.5 (2) |
| | TCC | 23.8 (5) | 28.6 (6) |
| | TCG | 33.3 (7) | 14.3 (3) |
| | TCT | 0 (0) | 14.3 (3) |
| Ile | ATA | 0 (0) | 0 (0) |
| | ATC | 55.6 (10) | 72.2 (13) |
| | ATT | 44.4 (8) | 27.8 (5) |
| Met | ATG | 100 (11) | 100 (11) |
| Gin | CAA | 31.8 (7) | 40.9 (9) |
| | CAG | 68.2 (15) | 59.1 (13) |
| His | CAT | 33.3 (3) | 33.3 (3) |
| | CAC | 66.7 (6) | 66.7 (6) |
| Pro | CCA | 6.9 (2) | 24.1 (7) |
| | CCC | 27.6 (8) | 31.0 (9) |
| | CCG | 51.7 (15) | 24.1 (7) |
| | CCT | 13.8 (4) | 20.7 (6) |
| Leu | CTA | 2.2 (1) | 0 (0) |
| | CTC | 17.4 (8) | 34.8 (16) |
| | CTG | 56.5 (26) | 32.6 (15) |
| | CTT | 4.3 (2) | 15.2 (7) |
| | TTA | 0 (0) | 0 (0) |
| | TTG | 19.6 (9) | 17.4 (8) |
| Glu | GAA | 58.3 (7) | 16.7 (2) |
| | GAG | 41.7 (5) | 83.3 (10) |
| Asp | GAC | 71.4 (25) | 74.3 (26) |
| | GAT | 28.6 (10) | 25.7 (9) |
| Ala | GCA | 6.9 (4) | 12.1 (7) |
| | GCC | 56.9 (33) | 36.2 (21) |
| | GCG | 31.0 (18) | 24.1 (14) |
| | GCT | 5.2 (3) | 27.6 (16) |
| Gly | GGA | 0 (0) | 13.5 (5) |
| | GGC | 73.0 (27) | 48.6 (18) |
| | GGG | 8.1 (3) | 18.9 (7) |
| | GGT | 18.9 (7) | 18.9 (7) |
| Val | GTA | 11.4 (4) | 0 (0) |
| | GTC | 34.3 (12) | 40 (14) |
| | GTG | 48.6 (17) | 40 (14) |
| | GTT | 5.7 (2) | 20 (7) |
| Tyr | TAC | 75 (6) | 87.5 (7) |
| | TAT | 25 (2) | 12.5 (1) |
| Cys | TGC | 66.7 (6) | 77.8 (7) |
| | TGT | 33.3 (3) | 22.2 (2) |
| Trp | TGG | 100 (3) | 100 (3) |
| Phe | TTC | 72.7 (8) | 81.8 (9) |
| | TTT | 27.3 (3) | 18.2 (2) |

### Example 2

### Preparation of mG2-aroA Transgenic Maize

I. Construction of Recombinant Expression Vector pS3300-UMG2

In order to increase expression level of mG2-aroA gene (SEQ ID NO. 2) in receptor organisms, when construct recombinant expression vector of mG2-aroA gene, we added Ω sequence and Kozak sequence at 5′ terminus of mG2-aroA gene. The Ω/Kozak sequence (briefly referred as to OMK) is shown as SEQ ID NO. 6 in the sequence listing. Ω sequence is derived from a translation enhancer of coding region of plant virus capsid protein gene, consisting of 67 bp TTAAC-rich sequence. There is a UAUUUUUACAACAA (SEQ ID NO. 12) sequence and 4 UUAC sequences at 5′ terminus, those sequences comprise ribosomes and rRNA binding site during translation of proteins synthesis. Kozak sequence is one that facilitates translation of exogenous genes in plant cells and that encodes ribosome binding protein. The promoter is a constitutive, Ubi promoter having sequence is shown in the SEQ ID NO. 5 of the sequence listing. Furthermore, at 3′ terminus of encoding SEQ ID NO. 2 consecutive termination codons are designed, and an synthetic PolyA+T-NOS stable terminator sequences are added. The PolyA+T-NOS sequence is shown in SEQ ID NO. 8 of the sequence listing. Wherein, PolyA can maintain the stability of mRNA, and the T-NOS terminator sequence ensure correct termination of translation. Further, according to mechanism of G2-aroA gene, the chloroplast transit peptide CTP is added prior to 5′ start codon ATG, such that proteins (i.e., G2-aroA protein) expressed by the target gene can be transported into chloroplast. Then, the resistance of mG2-aroA gene can be functioned better. The sequence of CTP is shown as SEQ ID NO. 7 in the sequence listing.

Construction of recombinant expression vector pS3300-UMG2 carrying mG2-aroA gene was specifically performed as follows:

1. Construction of an intermediate vector pUC19-UG2

a. the Ubi promoter (SEQ ID NO. 5) is synthetic, and the Pst I restriction sites are added at both terminus; after dephosphorylation of Pst I enzyme digestion pUC19 plasmids (purchase from Beijing Tian Enze Genetic Technology Limited Company, product No. 90202), connected with Ubi promoter fragment to obtain the pUC19-Ubi.

b. G2-aroA gene (SEQ ID NO. 1) is synthetic, and the BamH I and Kpn I restriction sites are added at both terminus; then connecting pUC19-Ubi obtained in step 1a by BamH I and Kpn I enzyme digestion to obtain the pUC19-Ubi-G2.

c. the PolyA+T-NOS terminator sequence (SEQ ID NO. 8, wherein positions 216-491 is a T-NOS terminator sequence) is synthetic, and the Kpn I and EcoR I restriction sites are added at both terminus; connecting pUC19-Ubi-G2 obtained in setp 1b by Kpn I and EcoR I enzyme digestion to obtain the pUC19-Ubi-G2-polyA-T-NOS.

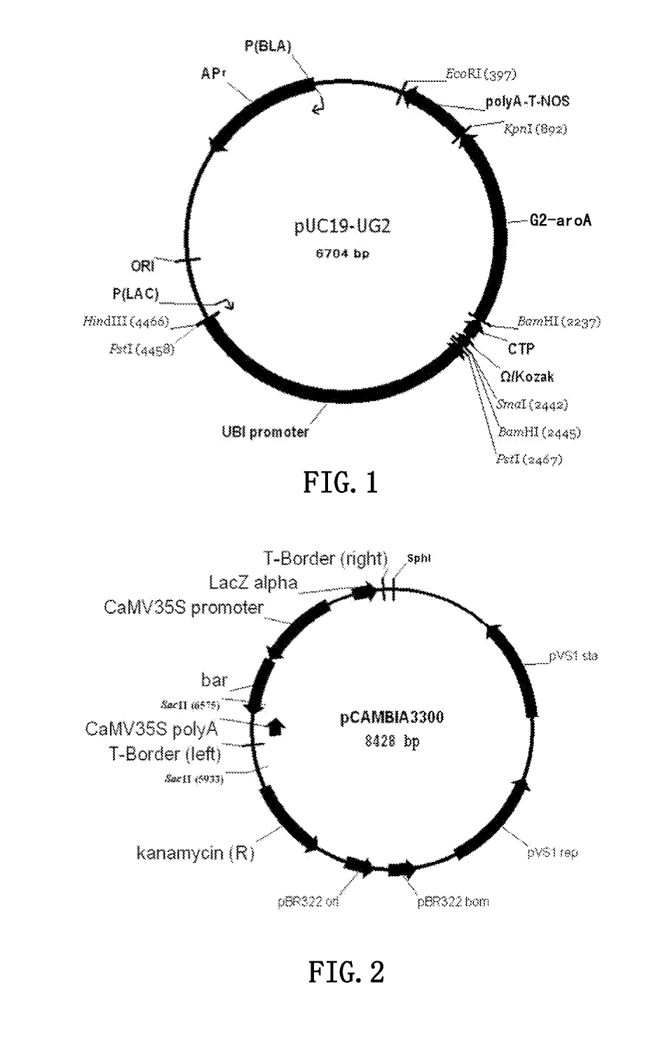

d. the OMK sequence (SEQ ID NO. 6) +CTP sequence (SEQ ID NO. 7) are synthetic, and the BamH I site is added at both terminus; then obtained pUC19-Ubi-G2-polyA-T-NOS in step 1c is digested by BamH I; after dephosphorylation connecting OMK-CTP (direct connection between SEQ ID NO. 6 and SEQ ID NO. 7) to obtain pUC19-UG2 (plasmid map, see FIG. 1).

2. Preparation of pS3300 vector by modification of pCAMBIA3300 vector

a. The expression vector pCAMBIA3300 (plasmid map, see FIG. 2) purchase from Beijing Dinguo changsheng Biotechnology Limited Company (CA: MCV038) was digested by two restriction endonuclease Sph I, Sac II, and then the large fragment was purified and recovered.

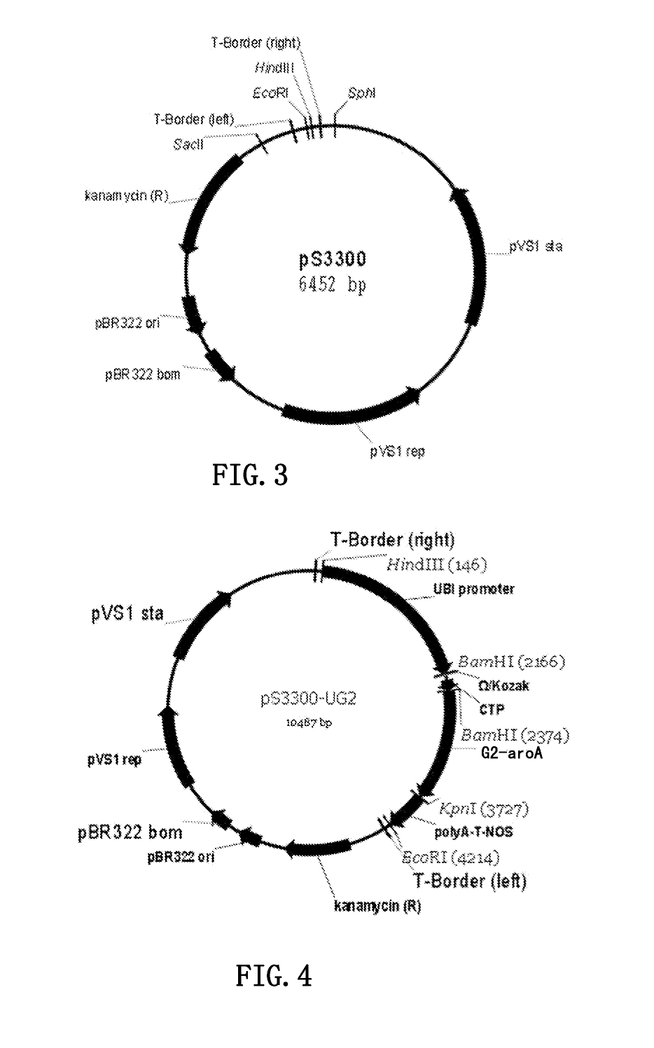

b. The gene fragment shown in SEQ ID NO. 4 of the sequence listing (consisting of from 5′ terminus, right boundary sequence of T-Border, connecting sequence and left boundary sequence of T-Border; in the connecting sequence, from 5′ terminus it contains sequences that recognized by Hind III and EcoR I restriction sites) was synthetic, and digested by restriction endonuclease Sph I, Sac II, and connected to large fragment recovered in step a to obtain a pS3300 vector (plasmid map, see FIG. 3).

3. Construction of recombinant expression vector pS3300-UMG2 carrying the mG2-aroA gene

a. The pS3300 vector obtained in step 2b was digested with two Hind III and EcoR I enzymes, then purification and recovery were performed.

b. The expression vector pUC19-UG2 (plasmid map, see FIG. 1) was digested with two Hind and EcoR I enzymes; the small fragment (Ubi-OMK-CTP-G2-polyA-T-NOS) was recovered; then connected to product obtained in step 3a to obtain the recombinant expression vector pS3300-UG2 carrying the G2-aroA gene (plasmid map, see FIG. 4).

c. The pS3300-UG2 vector obtained in step b was digested with two BamH I and Kpn I enzymes; and then the pS3300-Ubi-polyA-T-NOS and OMK+CTP fragments were recovered, respectively.

d. The newly synthesized mG2-aroA gene (SEQ ID NO. 3) carrying BamH I and Kpn I restriction sites at both terminus was digested with two BamH I and Kpn I enzymes; then connected to large pS3300-Ubi-polyA-T-NOS fragment obtained in above step c to obtain recombinant vector pS3300-Ubi-mG2-polyA-T-NOS.

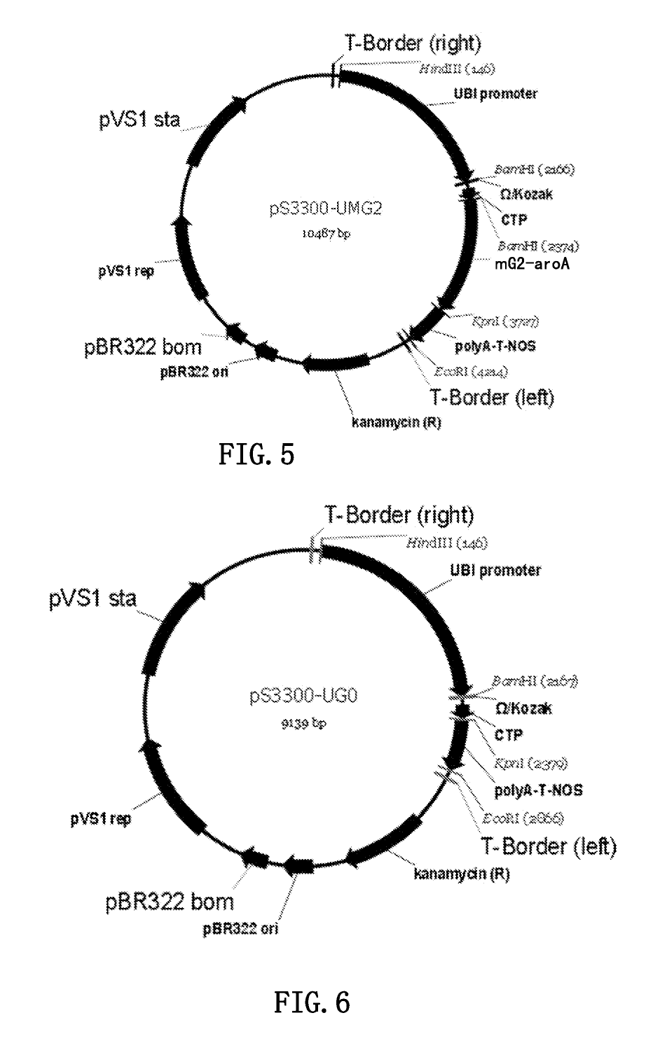

e. The recombinant vector pS3300-Ubi-mG2-polyA-T-NOS obtained in above step d was digested with BamH I enzyme; then after dephosphorylation, the product was connected to the OMK+CTP sequences obtained in above step d to obtain recombinant expression vector pS3300-UMG2 carrying the whole open reading frame of mG2-aroA gene (plasmid map, see FIG. 5). The recombinant expression vector pS3300-UMG2 contains a expression cassette having sequence is shown as SEQ ID NO. 10 in the sequence listing. Since the carried element, the expression cassette was designated as Ubi-OMK-CTP-mG2aroA-PolyA-T-NOS. The nucleotide sequence of the recombinant expression vector pS3300-UMG2 is shown as SEQ ID NO. 11 in the sequence listing.

4. As for construction of recombinant expression vector pS3300-UG2 of the initial gene G2-aroA (SEQ ID NO. 1), reference can be made to 3a and 3b.

5. Construction of pS3300-UG0 blank vector (control)

a. The OMK+CTP was amplified with the pS3300-UMG2 vector obtained in above step 3e as a templet, and the following primers were used:

OMK+CTP_F: 5′-GGATCCTATTTTTACAACAATTA-3′ (SEQ ID NO. 13) (the BamH I restriction site recognization sequence was underlined);

OMK+CTP_R: 5′-GGTACCTTCCGCCGTTGCTGAC-3′ (SEQ ID NO. 14) (the Kpn I restriction site recognization sequences was underlined).

b. After the amplification product was digested with two BamH I and Kpn I enzymes, the large pS3300-Ubi-polyA-T-NOS fragment obtained in above step 3c was connected to obtain the blank vector pS3300-UG0 (plasmid map, see FIG. 6).

II. Preparation of Transgenic Maize by Transforming Maize with the Recombinant Expression Vector

1. Preparation of Start Materials for Maize Transformation

Peeling the bract of young spike 9-13 days later after pollination of maize cultivar zong 31 (fine maize inbred line exhibition zong 3 and zong 31. maize since, 2009 (5)), and sterilizing its surface. Taking the immature embryo from sterilized young spike and placing in infection medium (formulation, see Methods in Molecular Biology, vol. 343: Agrobacterium Protocols, 2/e, volume 1)), washing 1-3 times and standing by service.

2. Transformation of Agrobacterium tumefaciens with the Recombinant Expression Vector

The recombinant expression vector pS3300-UMG2 or pS3300-UG2 was used to transform Agrobacterium tumefaciens LBA4404 (reference: Methods in Molecular Biology, vol. 343: Agrobacterium Protocols, 2/e, volume 1). At the same time, the Agrobacterium tumefaciens LBA4404 transformed with pS3300-UG0 blank vector was used as a control.

The Agrobacterium tumefaciens LBA4404 that has been transformed with recombinant expression vector pS3300-UMG2 via identification was designated as LBA4404/pS3300-UMG2; and the Agrobacterium tumefaciens LBA4404 that has been transformed with the recombinant expression vector pS3300-UG2 was designated as LBA4404/pS3300-UG2; the Agrobacterium tumefaciens LBA4404 that has been transformed with pS3300-UG0 blank vector was designated as LBA4404/pS3300-UG0.

3. Transformation of the Maize Immature Embryo with Agrobacterium tumefaciens

The immature embryo that has been washed in infection medium in step 1 was placed in three Agrobacterium tumefaciens solutions prepared in above step 2 with OD₆₀₀ of about 0.3-0.5 for 5 min; then immature embryo was transferred to co-culture medium (reference: Methods in Molecular Biology, vol. 343: Agrobacterium Protocols, 2/e, volume 1). Co-culturing for 3 days at about 20° C. under dark condition. The immature embryo not transformed with Agrobacterium tumefaciens was used as a control.

4. Preparation of Transgenic Maize Regeneration Plant

The co-cultured immature embryos in above step 3 were transferred to selective medium (reference: Methods in Molecular Biology, vol. 343: Agrobacterium Protocols, 2/e, volume 1). The glyphosate added in selective medium with a final concentration of 1 mM was used as a selection pressure. The transformed materials were cultured and screened. Every two weeks a subculture was done until crisp, cadmium yellow and vigorous resistant callus appeared.

The obtained resistant callus was transferred to inducible medium (reference: Methods in Molecular Biology, vol. 343: Agrobacterium Protocols, 2/e, volume 1) to induce its differentiation. 1 month later, the mature embryoid can be obtained. Then the embryoids were transferred to MS medium to root, and the T₀ generation transgenic maize regeneration plants were obtained. T₁ generation transgenic maize seeds were obtained after T₀ generation transgenic maize maturation. T₁ generation transgenic maize seeds continued to self-crossing and produce the T₂ generation transgenic maize seeds. The T₆ generation transgenic maize seeds can be obtained as described above. After seeding the T₆ generation transgenic maize seeds, the T₆ generation transgenic maize plants can be obtained.

5. Identification of T₆ Generation Transgenic Maize Plants

The PCR identification of T₆ generation transgenic maize plants was performed as follows:

Firstly, extracting genome DNAs of T₆ generation transgenic maize plants (plants transformed with mG2-aroA gene, plants transformed with G2-aroA gene, and plants transformed with pS3300-UG0 blank vector), respectively, as follows:

1) Taking 0.1-0.2 g young leaves of transgenic maize regeneration plants, grinding in liquid nitrogen, and then transferred to 1.5 ml of Eppendorf tube;

2) Adding 0.7 ml of CTAB solution (Tris with final concentration of 100 mM, NaCl with final concentration of 1.4M, EDTA with final concentration of 20 mM, CTAB with final concentration of 2% (w/v), mercaptoethanol with final concentration of 0.1% (v/v)); blending by reversion once every 10 min for 45 min at 60° C.

3) Adding 0.7 ml of phenol:chloroform (volume ratio is 1:1), and reversing for several times; then centrifugalization at 1000 rpm for 5 min; and the supernatant was transferred to a new centrifugal tube. Adding 0.7 ml of chloroform:isoamyl alcohol (volume ratio is a 24:1), and blending; then centrifugalization at 1000 rpm for 5 min, and the supernatant was transferred to a new centrifugal tube.

4) Adding 0.7 ml of isopropyl alcohol into the centrifugal tube, and blending by reversion; then centrifugalization at 1000 rpm for 10 min; and the supernatant was discarded; washing once with 70% alcohol, and drying by vacuumization; then dissolved in 50 μL of sterile water for PCR detection.

Secondly, the genome DNAs of T₆ generation transgenic maize plants (plants transformed with mG2-aroA gene, plants transformed with G2-aroA gene, and plants transformed with pS3300-UG0 blank vector) prepared above were used as templets for PCR identification as follows:

1) the initial gene (G2-aroA) identification was performed for 11 T₆ generation transgenic maize plants transformed with G2-aroA genes; the T₆ generation transgenic maize plants transformed with pS3300-UG0 blank vector were used as a negative control 1, non-transgenic maize were used as a negative control 2, and reaction systems without any templets added were used as a blank control. PCR amplification primers are as follows:

Forward primers: CGGCTCCAAATCCATTACCAA (SEQ ID NO. 15; positions 147-167 of SEQ ID NO. 1)

Reverse primers: GCCACTTCAATCGGCGCTTC (SEQ ID NO. 16; reverse complementary sequences of positions 631-650 of SEQ ID NO. 1)

The reaction system (20 μL): DNA 1 μL, (20-50 ng); 10× buffer 2 μL; MgCl₂ (2.5 mM) 2 μL; dNTP (2.5 mM) 2 μL; Taq enzyme 0.2 μL; primers 10 μM; adding sterile water to 20 μL. The reaction condition: 94° C. 5 min; 94° C. 45 s, 55° C. 45 s, 72° C. 1 min, 35 cycles; 72° C. extension 5 min; and the length of amplification product was 610 bp.

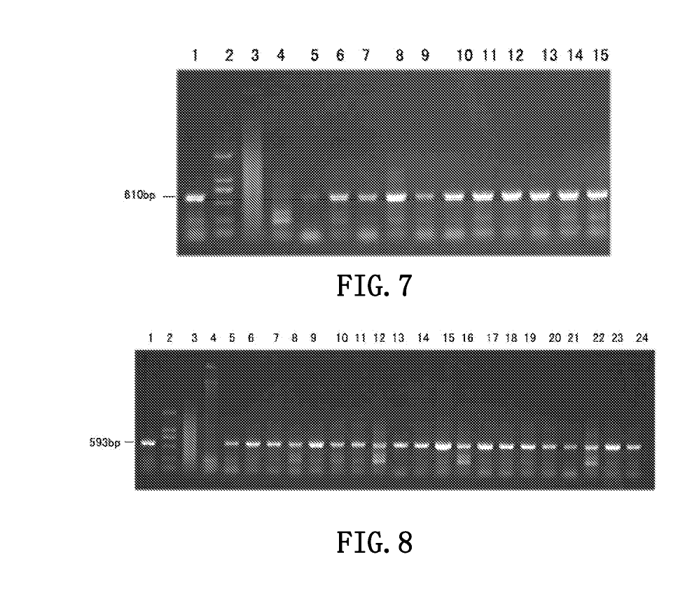

The result of identification is shown in FIG. 7. 11 T₆ generation transgenic maize plants transformed with G2-aroA gene shown in lanes 5-15 all show 610 bp size of target band obtained by amplification; neither the negative control group (negative control 1 and negative control 2) nor blank control group shows amplified target band. The result demonstrates that G2-aroA gene has been integrated into genome of T₆ generation G2-aroA transgenic maize.

2) The optimized gene (mG2-aroA) was identified for 20 T₆ generation transgenic maize plants transformed with mG2-aroA gene; the T₆ generation transgenic maize plants transformed with pS3300-UG0 blank vector were used as a negative control 1; the non-transgenic maize plants were used as a negative control 2; and the reaction systems without any templet added were used as a blank control.

Forward primers: CCACCTGGCTCCAAGTCTATCA (SEQ ID NO. 17; positions 142-163 of SEQ ID NO. 2)

Reverse primers: GCGTCAACCTGTGCTCCAAA (SEQ ID NO. 18; reverse complementary sequences of positions 715-743 of SEQ ID NO. 2)

The reaction system is the same as that described above. The reaction condition: 94° C. 5 min; 94° C. 45 s, 55° C. 45 s, 72° C. 45 s, 35 cycles; 72° C. extension 5 min; the amplification product length 593 bp.

The result of identification is shown in FIG. 8. The 20 T₆ generation transgenic maize plants transformed with mG2-aroA gene shown in lanes 5-24 all show 593 bp size of target band obtained by amplification; neither the negative control group (negative control 1 and negative control 2) nor the blank control group shows amplified target band. The result demonstrates that mG2-aroA gene has been integrated into genome of T₆ generation mG2-aroA transgenic maize.

### Example 3

### The Double-Antibody Sandwich ELISA Detection of Expression of Transgenic Maize Plants G2-aroA Protein

The sample extracting solution: 25 mM of Tris-Cl (pH8.0), 10 mM of KCl, 20 mM of MgCl₂.6H₂O, 1 mM of DTT, 1 mM of PMSF (added before using).

The coating buffer: taking 1.5 g of Na₂CO₃, 2.93 g of NaHCO₃, and set to 1000 mL with distilled water, pH9.6.

The washing solution (PBST): taking 1 mL of Tween 20, adding phosphate buffer (PBS) set to 1000 mL, pH7.5;

The phosphate buffer (PBS): taking 8.0 g of NaCl, 0.2 g of KH₂PO₄, 2.96 g of Na₂HPO₄.12H₂O, adding 1000 mL of distilled water, pH7.5.

The sample buffer (PBST): taking 1 mL of Tween 20, adding phosphate buffer (PBS) set to 1000 mL, pH7.5;

The phosphate buffer (PBS): taking 8.0 g of NaCl, 0.2 g of KH₂PO₄, 2.96 g of Na₂HPO₄.12H₂O, adding 1000 mL of distilled water, pH 7.5.

The substrate buffer: taking 0.1 g of MgCl₂ or 0.2 g of MgCl₂.6H₂O, 97.0 mL of diethanolameine, dissolving in 1000 mL of distilled water, pH9.8, and storing at 4° C.

The termination buffer: 3 mol/L of NaOH, pH 12.0.

The blocking solution: taking 3 g of bovine serum albumin (BSA) and dissolving in 100 mL of the coating buffer.

The coating buffer (carbonate coating buffer): taking 1.5 g of Na₂CO₃, 2.93 g of NaHCO₃, and set to 1000 mL with distilled water, pH9.6.

1. Treatment of Samples

Taking T₆ generation transgenic maize plants (including plants transformed with the mG2-aroA gene and plants transformed with G2-aroA gene) at the same growth state (five-leaf stage), and taking about 1 g (fresh weight) of each function leaf (top-three leaves) plates, then grinding in liquid nitrogen. Transferring into 10 ml of centrifugal tube, into which 3 ml of sample extracting solution was added, and shaking strongly. Then centrifugalizing for 1 h at 4° C. The supernatant was taken as a sample to be detected and stand by service. Since the transformed genes are different, there are two samples to be detected, that is, a sample derived from mG2-aroA transgenic plants (sample to be detected is mG2-aroA) and a sample derived from G2-aroA transgenic plants (sample to be detected is G2-aroA). At the same time, the T₆ generation maize plants transformed with pS3300-UG0 blank vector and the non-transgenic maize plants were used as controls.

The weight weighting when the leaf plates were ground in liquid nitrogen into powder samples and added into centrifugal tube containing the extracting solution subtracts that weighting before adding sample into centrifugal tube containing the extracting solution to obtain the fresh weight described above.

2. Detection of the Samples

The detection of the samples was performed as follows:

(1) Coating: the primary antibody (rabbit antibody) with concentration of 1.5 mg/ml (preparation method, see last part of this specification) against G2-aroA protein was diluted in coating buffer. And then after dilution added into ELIAS plate with a volume ratio of 1:1000 between polyclonal antibody against G2-aroA protein and coating buffer, 100 μL per well, and coating overnight in wet box at 4° C.

(2) Washing plate: the coating solution was removed, and then washed 5 times with washing solution in plate washer.

(3) Blocking: 120 μL of blocking solution was added into each well, and then the well was incubated 1 h in a wet box at 37° C., and the blocking solution was removed.

(4) Reaction: in one aspect, G2-aroA protein standard (preparation method, see last part of this specification) was diluted to concentration of 10 μg/mL with the sample dilution solution; then 11 dilution gradients with 2-fold dilution (including the 0 well) were obtained, repeating 3 times, and each well contains 100 μL. Wherein the 0 well (without standard solution added instead of 100 μL of sample dilution solution) was used as a control well, other 10 gradient wells were used as experiment wells. Then the well was incubated for 1 h at 37° C. Finally, the standard curve was created.

In another aspect, the sample to be detected prepared in step 1 was diluted with sample buffer in a ratio of 1:5, and then 100 μL of diluted sample was added to each well. Then the well was incubated for 1 h at 37° C. for detection.

(5) Washing plate: see above step (2).

(6) Adding of the enzyme labeled antibody: the enzyme labeled antibody (murine antibody) against G2-aroA protein (preparation method, see last part of this specification) was diluted with sample dilution solution in a ratio of 1:1000, after mixing 100 μL of diluted sample was added into each well. Then the well was incubated for 1 h at 37° C.

(7) Washing plate: see above step (2).

(8) Adding substrate for coloration: weighting 30 mg of p-nitrophenyl phosphate disodium (PNPP) and adding into 30 ml of substrate buffer (which must be used in 10 min after preparation); then 100 μL of solution above was added into each well; after 20 min 50 μL of termination buffer was added to terminate the reaction.

(9) Measurement: the OD value was measured at 405 nm.

(10) Plotting the standard curve: the standard solution concentration of G2-aroA protein of various of concentration (ng/mL) was plotted on X axis, and the OD value of G2-aroA protein standard measured in step (9) was plotted on Y axis, then the standard curve was plotted by EXCEL.

(11) The OD value of sample to be detected measured in step (9) was used in standard curve equation obtained in step (10) to calculate the content of G2-aroA protein in sample to be detected.

The content of G2-aroA protein in fresh weight=the content of G2-aroA protein of sample to be detected×the volume of sample to be detected/fresh weight of the sample, unit: μg/g.

The experiments were repeated for 3 times, and the average values of the three experiments was used to obtain the standard curve (FIG. 10), wherein the standard curve equation is y=9035.2×+198.75 (R²=0.9997).

The detection result of sample to be detected is shown in Table 2. The expression amount of G2-aroA protein in T₆ generation mG2-aroA transgenic maize and T₆ generation G2-aroA transgenic maize in remarkably higher than transgenic maize transformed with blank vector (average 0.674 μg/g). Furthermore, the expression amount of G2-aroA protein by optimized G2-aroA gene (mG2-aroA) in T₆ generation transgenic maize (average 15.057 μg/g) is far higher than expression amount of G2-aroA protein by initial G2-aroA gene (G2-aroA) in T₆ generation transgenic maize (average 3.735 μg/g). The expression amount of G2-aroA protein in non-transgenic maize plants and the T₆ generation maize plants transformed with pS3300-UG0 blank vector are substantially the same, there is no significant difference. The result demonstrates that after codons optimization, the expression amount of G2-aroA protein in transgenic maize is significantly increased.

TABLE 2
ELASA detection result of expression of G2-aroA protein in
transgenic plants

| | G2-aroA | | G2-aroA | | G2-aroA |
|---|---|---|---|---|---|
| sample to be | protein | sample to | protein | | protein |
| detected- | concentration | be detected- | concentration | blank vector | concentration |
| mG2-aroA | (μg/g) | G2-aroA | (μg/g) | control | (μg/g) |
| 1 | 4.339 | 1 | 15.096 | 1 | 0.893 |
| 2 | 3.304 | 2 | 10.671 | 2 | 0.586 |
| 3 | 2.977 | 3 | 20.227 | 3 | 0.722 |
| 4 | 4.321 | 4 | 14.237 | 4 | 0.497 |
| average | 3.735 | average | 15.057 | average | 0.674 |

It to be noted that numbering 1, 2, 3 and 4 represent sample to be detected of four different plants, respectively; G2-aroA protein concentration unit μg/g represents microgramme of G2-aroA protein detected in per gram of leaf plate (fresh weight).

### Example 4

### Detection of Herbicide Resistance of Transgenic Maize Plants in Field

1. the experimental materials: T₆ generation transgenic maize plants, including plants transformed with mG2-aroA gene, plants transformed with G2-aroA gene, and plants transformed with pS3300-UG0 blank vector. At the same time, the non-transgenic maize plants were used as a control.

2. experiment design: 5M of row length, 3 row plots, 3 times of repeat, density: 60×35 cm

3. experiment treatment: applying Roundup (containing 41% of glyphosate, recommbinated applying amount in field is 150-250 ml/mu) in an amount of 800 ml/mu.

4. the experiment treatment timing: 5-6 leaf age. The experimental result was observed 7 days later.

5. experiment result

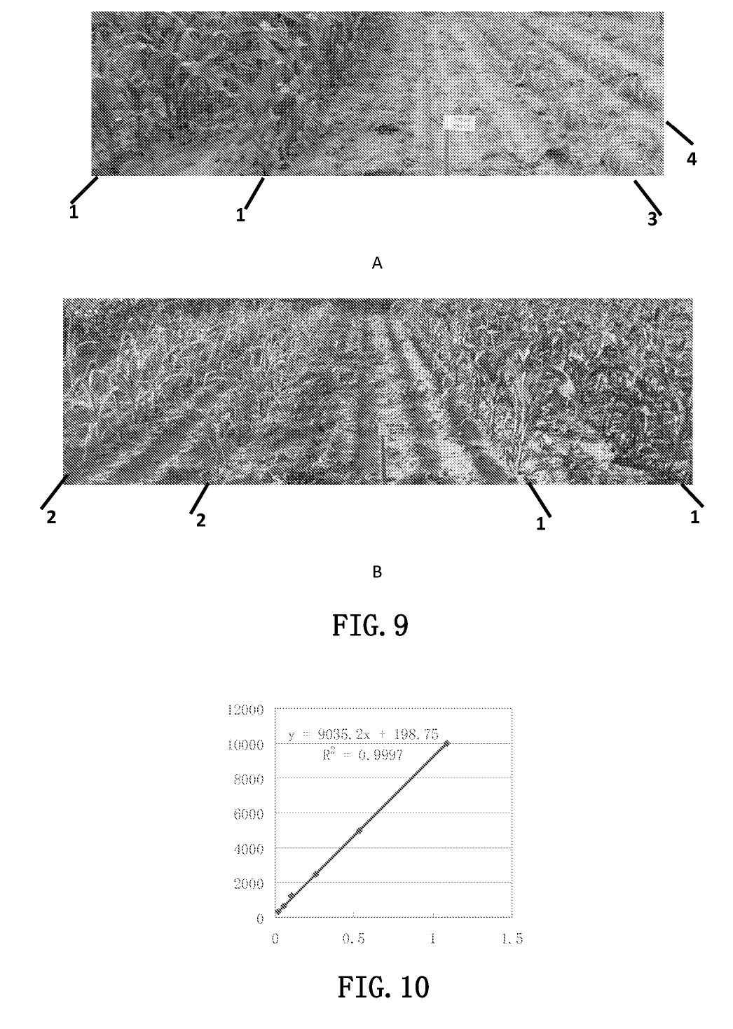

As shown in FIG. 9 (15 days after applying Roundup): (1) as for plants transformed with G2-aroA gene, after 800 ml/mu Roundup treatment, they suffered from very serious phytotoxicity, and all plants showed yellowing, abnormalities. At late stage of plants growth, most of plants showed that the tassels did not differentiate, and ears did not silk or plants were short; (2) as for plants transformed with mG2-aroA gene, after 800 ml/mu Roundup treatment, phytotoxicity was not obvious, they grew normally. ++At late stage of plants growth, there was no phytotoxicity response; (3) as for plants transformed with pS3300-UG0 blank vector and maize plants transformed with non-transgenic, the phytotoxicity was very serious, all plants were dead. The result demonstrates that the glyphosate resistance of maize transformed with codon optimized mG2-aroA gene increase significantly.

I. Method for preparing primary antibody (rabbit antibody) against G2-aroA protein in Example 3 is as follows:

The purified G2-aroA protein (preparation method, see step II below) was used as an immunogen to immunize New Zealand White Rabbits with body weight of about 2 kg. Polyclonal antibodies against G2-aroA protein were obtained after serum extraction and antibody purification step as follows: preparing immunogens via the same method as that used in step I of Example 1; immunizing New Zealand White Rabbits with a body weight of about 2 kg by obtained immunogen; isolating the serums to prepare polyclonal antibodies against G2-aroA protein. The polyclonal antibodies against G2-aroA protein can be used in double-antibody sandwich as coating antibodies to detect G2-aroA protein.

II. The method for preparing G2-aroA protein standard in Example 3 is as follows:

The DNA fragment shown in SEQ ID NO. 1 was connected to polyclonal sites of prokaryotic expression vector pET-28a (+) to obtain prokaryotic expression vector pET-28a-G2-aroA that expresses the G2-aroA protein. The pET-28a-G2-aroA was transformed into Escherichia coli BL21. After induction with IPTG, it expressed G2-aroA protein. The myceliums were collected, and broken by ultrasonic. All expressed proteins were released into the extracting solution. Then the His label protein purification kit (purchase from Beijing kangwei shiji biotechnical Limited Company, cat. no. CW0009A) was used for purification. After purification G2-aroA protein was detected through SDS-PAGE electrophoresis.

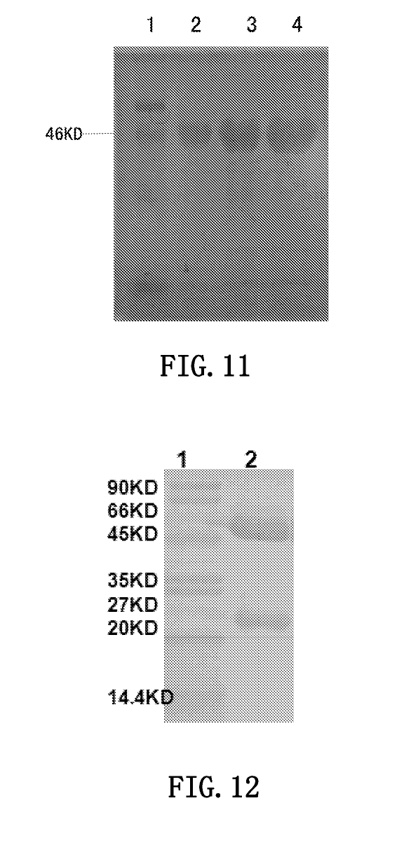

SDS-PAGE electrophoresis identification result is shown in FIG. 11. The purity is up to 95% based on page identification. The concentration of G2-aroA protein is up to 1.3 mg/ml according to eppendorf proteins nucleic acid detector biophotometer plus. The purified G2-aroA protein is the immunogen 5780.

The amino acid sequence of G2-aroA protein is shown as SEQ ID NO. 9 in the sequence listing.

III. Method for preparing enzyme labeled antibody (murine antibody) against G2-aroA protein in Example 3 is as follows:

Balb/C mice: Beijing weitong lihua experimental animals Limited Company

Sp2/0: Beijing kangwei shiji biotechnical Limited Company

1. Preparation of Immunogen 5780

The method is the same as “method for preparing G2-aroA protein standard in step II of Example 3”. The purified G2-aroA protein is the immunogen 5780. The amino acid sequence of G2-aroA protein is the SEQ ID NO. 9 in the sequence listing.

2. Animals Immunization

5 female Balb/C mice of 6 week old were used as experimental animals, and immunized according to following protocol and scheme:

a. pre-immunization: collecting serum by tail cutting bleeding, as a negative control.

b. primary immunization: after the G2-aroA protein solution with concentration of 0.64 μg/μl was filtered by sterile filter, and equivalent volume of complete Freund's adjuvant was added. Then the magnetic stirrer was used to fully stir and emulsify until it did not disperse when dip into water, so as to obtain the immunogen. The emulsified immunogen was used to immunize Balb/C mice by multiple point injection under back skin. The dosage for each mouse was 80 μg of G2-aroA protein (250 μl of emulsified immunogen).

c. The first booster immunization: 21 days later after first immunization, the G2-aroA protein solution with concentration of 0.32 μg/μl was filtered by sterile filter, and equivalent volume of incomplete Freund's adjuvant was added. Then the magnetic stirrer was used to fully stir and emulsify until it did not disperse when dip into water, so as to obtain the immunogen. The emulsified immunogen was used to immunize Balb/C mice by multiple point injection under back skin. The dosage for each mouse was 40 μg of G2-aroA protein (250 μl of emulsified immunogen).

d. The second booster immunization: 14 days later after first booster immunization, the second booster immunization was performed. The method is the same as the first booster immunization in step c.

e. The final booster immunization: 14 days later after the second booster immunization, the G2-aroA protein solution with concentration of 0.8 μg/μl was filtered by sterile filter to obtain the immunogen. The obtained immunogen was injected in spleen as a booster immunization to Balb/C mice. The dosage for each mouse was 80 μg of G2-aroA protein (100 μl of emulsified immunogen).

3. Cell Fusion and Cloning

14 days later after the second booster immunization, the serum was collected by tail cutting bleeding, and the serum titer was measured by indirect ELISA (S780 was used as a coating antigen). 3 days later after final booster immunization in above step, the mouse that has the best serum titer measured by above indirect ELISA (titer is 1:720000) was selected to take spleen cells. Then the spleen cells and SP2/0 myeloma cell in 9:1 by number were fused by PEG (PEG4000) common fusion method.

At third and sixth day after fusion, the liquids were replaced. At seventh day the indirect ELISA (S780 was used as a coating antigen) was used to screen supernatant of fusion cells. The positive cell wells were selected and cells therein were transferred to 24-well culture plate for amplification culturing. When cells grew up to ⅓ field of microscope, cells' supernatants were collected. The indirect ELISA (S780, K8 (Mosanto glyphosate resistant materials mon810 extracting solution), positive control (glyphosate resistant materials extracting solution), negative control (non-transgenic materials extracting solution) were used as coating antigens, respectively) was used to perform a secondary screen. Those cells reacting actively with 5780 and positive control, while no reacting with K8 and negative (bold 5F11 in Table 3) were selected. The limiting dilution was used to conduct subcloning. After 3 times subcloning, finally obtained monoclonal hybridoma cell strains that stably secrete anti-G2-aroA protein, and designated as AntiG2-5F11. The hybridoma cell strain has been deposited in China General Microbiological Culture Collection Center (simplified as CGMCC, address: Institute of Microbiology Chinese Academy of Sciences, NO. 1-3 West Beichen Road, Chaoyang District Beijing China, postal code: 100101), retrieving number: CGMCC No. 5772.

Above K8 (Mosanto glyphosate resistant materials mon810 extracting solution), positive control (our glyphosate resistant materials extracting solution), negative control (non-transgenic materials extracting solution) used as coating antigens were prepared as follows: taking 0.3 g of sample materials (Mosanto glyphosate resistant materials mon810, or T₆ generation mG2-aroA transgenic maize, or non-transgenic maize cultivar zong 31 obtained in Example 2); after grinding in liquid nitrogen, transferred into 2 ml of centrifugal tube and 1 ml of sample extracting solution was added, and shaking strongly; extracting for 1 h at 4° C., centrifugalizing for 10 min at 12000 rpm, then taking supernatant to measure samples. Wherein, components of sample extracting solution is 25 mm of Tris-Cl (pH8.0); 10 mm of KCl; 20 mm of MgCl₂.6H₂O; 1 mm of DTT; 1 mm of PMSF (added before using).

The above indirect ELISA was conducted as follows:

1) Coating: 100 μL 2 μg/ml of antigen solution was added into 96-well ELIAS plate; at the same time, uncoated antigen was used as a control. Then coating overnight at 4° C., and washing 3 times with PBS buffer.

The above antigen solution can be 5780, K8, positive and negative solution.

2) Blocking: 150 μL of blocking solution (3% bovine serum proteins) was added into each well; incubating for 2 h at 37° C., and the blocking solution was discarded; then washing 3 times, drying by beating. Placing in freezer at 4° C. for storage and standing by service.

3) Adding of samples to be detected:

a. as for serum titer detection, the first well was diluted in 1:1000, and other wells were diluted in a 1:3 gradient compared to the latter well, then incubating for 30 min 37° C.; and washing plate for 4 times, drying by beating.

b. as for cell supernatant detection, taking 100 μl of cell supernatant into corresponding ELIAS plate, then incubating for 30 min at 37° C.; and washing plate for 4 times, drying by beating.

At the same time, serum/antibody/ascites/cell supernatants derived from unimmunized mice were used as control; the PBS was used to replace sample to be detected as a control (negative control well).

4) Adding of enzyme labeled secondary antibody: taking goat anti-mice IgG(H+L)-HRP, after dilution in 1:5000, 100 μl/well, incubating for 20 to 30 min at 37° C., washing 4 times, and drying by beating.

5) Coloration: 20×TMB was diluted to 1×TMB, 100 μl/well, coloration for 15-30 min at 37° C.

6) termination: 50 μl of termination solution (2M H₂SO₄) was added in each well.

7) Reading: the OD value of each well was measured at 450 nm single wave length, when the ratio of OD value of negative control well (the PBS was used to replace control sample to be detected, N) (P/N) is greater than 2.1, it is used as a critical point to determine a positive or finite titer.

Method for ELISA result determination: (1) when screening positive cells, if P/N>2.1, they are considered as positive cells; if 1<P/N<2.1, increasing coating concentration and then conducting next detection, if P/N>2.1, they are considered as positive. (2) when measuring titer, it is represented by largest dilution folds of serum (or ascites, antibody) that makes P/N>2.1.

TABLE 3
Result of rescreening to fusion cell supernatants using the
indirect ELISA

| coating | | | | | | | | | | | | |
|---|---|---|---|---|---|---|---|---|---|---|---|---|
| antigen | 1C2 | 1C11 | 1E6 | 2C1 | 2F7 | 3D12 | 3F9 | 4B11 | 5B2 | 5F11 | PBS | positive |
| S780 | 2.398 | 2.463 | 2.655 | 2.232 | 2.067 | 2.332 | 3.431 | 3.400 | 3.474 | 3.452 | 3.489 | 3.470 |
| K8 | 0.048 | 0.057 | 0.044 | 0.049 | 0.041 | 0.053 | 0.048 | 0.045 | 0.050 | 0.046 | 0.053 | 0.104 |
| positive | 0.297 | 0.162 | 0.398 | 0.289 | 1.424 | 0.245 | 1.762 | 0.920 | 0.393 | 2.427 | 0.068 | 1.291 |
| negative | 0.050 | 0.046 | 0.041 | 0.045 | 0.043 | 0.045 | 0.067 | 0.043 | 0.045 | 0.044 | 0.057 | 0.052 |

It to be noted that 1C2-5F11 represent screened positive cells by the indirect ELISA (S780 is the coating antigen) 7 day later after cell fusion. The larger absorbance demonstrates that the antibody has a higher affinity to the antigen.

4. Preparation and Purification of Monoclonal Antibodies

A. Increment Culturing

The hybridoma cell strain AntiG2-5F11CGMCC No. 5772 was placed on RPMI 1640 medium, culturing for 3 days at 37° C., and collecting cell supernatants. protein G affinity column (Pharmacia) was used to conduct purification as follows:

(1) Equilibrium: the protein G affinity column was balanced by connecting buffer to baseline and stable.

(2) Loading: loading the collected cell supernatants, and collecting flow through liquid; then loading flow through liquid on the column and continue to balance to baseline and stable.

(3) Elution: adding elution buffer elution, and elution peak.

(4) The elution peak was dialysed and collected with 0.01M of PBS (pH7.2), storing the purified antibody in 0.01M of PBS (pH7.2).

(5) Measuring concentration of purified monoclonal antibodies by protein quantification detector, Amersham Biosciences.

(6) SDS-PAGE detection purified antibody purity, antibody loading amount: 8 μg.

B. Preparation of Ascites

Each of 2 BALB/C mice was injected 0.5 ml of paraffin oil. 7 days later, hybridoma cell strain AntiG2-5F11 CGMCC No. 5772 was resuspend in serum free medium. The paraffin injected mice was injected in 1×10⁶ cells/0.5 ml per mice. 14 days later after cell injection, collecting ascites. The indirect ELISA (coating antigen is S780) was used to conduct ascites titer detection, and the specific process and the result determination method is the same as that describe in step 3 (wherein, the step 3) is conducted according to a). The result demonstrates that its titer was 1:81000. The protein G affinity column (Phamacia) was used to purify ascites. The specific process is the same as that described in step 1.

5. Identification of Monoclonal Antibodies

(1) Identification of Monoclonal Antibodies Subclass

With subclass detection kit produced by Southern Biotech (including Goat anti Mouse IgG1-HRP (1070-50), Goat anti Mouse IgG2a-HRP (1080-05), Goat anti Mouse IgG2b-HRP (1090-05), Goat anti Mouse IgG3-HRP (1100-05), Goat anti Mouse IgA-HRP (1040-05), Goat anti Mouse IgM-HRP (1020-05), Goat anti Mouse kappa-HRP (1050-05) and Goat anti Mouse Lambda-HRP (1060-05), totally 8 components), antibody subclass detection was conducted to monoclonal antibodies according to kit introduction, wherein the coating antigen is S780, concentration is 2 μg/ml, and the coating dosage is 100 μl, detection wavelength is 450 nm. The detection result is shown in Table 4. The heavy chain constant region of monoclonal antibodies is IgG1 subclass, and the light chain constant region is Lamda subclass.

TABLE 4
Subclass identification of monoclonal antibodies

| | IgG1 | IgG2a | IgG2b | IgG3 | IgM | IgA | Kappa | Lamda |
|---|---|---|---|---|---|---|---|---|
| Monoclonal | 2.252 | 0.051 | 0.048 | 0.058 | 0.055 | 0.056 | 0.065 | 0.461 |
| antibodies | | | | | | | | |

(2) Detection of Monoclonal Antibodies Titer

With the indirect ELISA (coating antigens were S780, K8, positive and negative controls, specifically as that in step 3), titer detection was conducted to the purified monoclonal antibodies obtained in step 4. The PBS solution replacing sample to be detected was used as a negative control. The specific process and the result determination method is the same as that describe in step 3 (wherein, the step 3) is conducted according to a).

The detection result is shown in Table 5. When the positive control was used as a coating antigen, the titer of monoclonal antibodies purified in ascites is 1:81000 (0.292/0.083=3.518>2.1).

TABLE 5
The detection result of monoclonal antibodies titer

| | coating | | | | | | | | |
|---|---|---|---|---|---|---|---|---|---|
| | antigen | 1:1000 | 1:3000 | 1:9000 | 1:27000 | 1:81000 | 1:240000 | 1:720000 | PBS |
| monoclonal | S780 | 3.501 | 3.333 | 2.729 | 1.707 | 0.706 | 0.239 | 0.117 | 0.056 |
| antibodies | K8 | 0.455 | 0.273 | 0.202 | 0.215 | 0.197 | 0.188 | 0.162 | 0.181 |
| purified | positive | 3.269 | 1.804 | 0.940 | 0.673 | 0.292 | 0.117 | 0.088 | 0.083 |
| from | negative | 0.420 | 0.185 | 0.117 | 0.174 | 0.099 | 0.103 | 0.122 | 0.072 |
| ascites | | | | | | | | | |

6. Purity Identification of Monoclonal Antibodies by SDS-PAGE

Purity identification of monoclonal antibodies obtained in step 4 was conducted by SDS-PAGE. The result is shown in FIG. 12. Two target bands were shown on gel, the larger one is heavy chain, the smaller one is the light chain. The target bands are clear no impurity band. It can be seen that the purified monoclonal antibodies has a better purity.

### INDUSTRY APPLICATION

The transgenic maizes having synthesized glyphosate-resistant gene (SEQ ID NO. 2) provided in present invention have a remarkably higher expression amount of G2-aroA protein as compared to transgenic maizes having initial G2-aroA gene (SEQ ID NO. 1). The expression amount of G2-aroA protein in per gram (fresh weight) of leaf plate can be up to 15.057 μg, far larger than 3.735 μg in G2-aroA gene transgenic maize. At the same time, the resistance to glyphosate by the transgenic maizes having synthesized glyphosate-resistant gene (SEQ ID NO. 2) provided in present invention is remarkably improved as compared to G2-aroA gene (SEQ ID NO. 1) having transgenic maize. As for T₆ generation plants transformed with mG2-aroA gene, at maize 5-6 leaf age stage, after applying 800 ml/mu Roundup, phytotoxicity was not obvious, they grew normally. At late stage of plants growth, there was no phytotoxicity response; as for T₆ generation plants transformed with G2-aroA gene, after 800 ml/mu Roundup treatment, phytotoxicity was very serious, plants all show yellowing, abnormalities. At late stage of plants growth, most of plants showed that the tassels did not differentiate, and ears did not silk or plants were short.

What is claimed is:

1. A DNA molecule, which is one of following (a)-(c):
(a) a DNA molecule having nucleotide sequence shown as sequence 2 in the sequence listing;
(b) a DNA molecule having nucleotide sequence shown as positions 1-1335 of sequence 2 in the sequence listing;
(c) a DNA molecule having nucleotide sequence that has an identity at least 98% with sequence 2 or positions 1-1335 of sequence 2 in the sequence listing, and encoding the protein shown in sequence 9.

2. An expression cassette, recombinant vector, recombinant host bacteria, recombinant cell line or transgenic plant containing the DNA molecule of claim 1.

3. The expression cassette according to claim 2, characterized in that the expression cassette includes elements of 1)-3) as follows:
1) a promoter; 2) the DNA molecule starting transcription by the promoter; 3) a transcription terminator sequence.

4. The recombinant vector according to claim 2, characterized in that the recombinant vector is a recombinant cloning vector or a recombinant expression vector.

5. The expression cassette according to claim 3, characterized in that the promoter is an Ubi promoter; the transcription terminator sequence is a sequence shown as positions 216-491 of sequence 8 in the sequence listing, or a sequence having an identity of at least 80% with positions 216-491 of sequence 8 and possessing transcription termination function.

6. The recombinant vector according to claim 4, characterized in that the promoter in the recombinant expression vector for starting the DNA molecule transcription is an Ubi promoter; the transcription terminator sequence in recombinant expression vector for terminating the DNA molecule transcription is a sequence shown as positions 216-491 of sequence 8 in the sequence listing, or a sequence having an identity of at least 80% with positions 216-491 of sequence 8 and possessing transcription termination function.

7. The expression cassette according to claim 5, characterized in that the sequence of Ubi promoter is the sequence 5 in the sequence listing, or a sequence having an identity of at least 80% with the sequence 5, and having the promoter function.

8. The expression cassette or the recombinant vector according to claim 2, characterized in that the expression cassette or the recombinant expression vector further include an OMK sequence; the OMK sequence consists of Ω sequence and Kozak sequence that connected in succession.

9. The expression cassette or the recombinant vector according to claim 8,
characterized in that the OMK sequence is the sequence 6 in the sequence listing, or a sequence having an identity of at least 80% with sequence 6 and possessing the enhancer function.

10. The expression cassette or the recombinant vector according to claim 2, characterized in that the expression cassette or the recombinant expression vector further include a chloroplast transit peptide sequence; the chloroplast transit peptide sequence is the sequence 7 in the sequence listing, or a sequence having an identity of at least 80% with sequence 7 and possessing signal peptide function.

11. The expression cassette or the recombinant vector according to claim 2, characterized in that the expression cassette consists of the Ubi promoter, the OMK sequence, the chloroplast transit peptide sequence, the glyphosate-resistant gene and the transcription terminator sequences connected in succession; the sequence of the expression cassette is specifically the sequence 10 in the sequence listing;
the sequence of the recombinant vector is the sequence 11 in sequence listing.

12. A RNA obtained by transcription of the DNA molecule according to claim 1.

13. A use of the DNA molecule according to claim 1, for breeding glyphosate resistant transgenic maize.

14. A use of the DNA molecule according to claim 1 for increasing maize G2-aroA protein expression amount; the G2-aroA protein is one shown as sequence 9 in the sequence listing.

15. A method for breeding glyphosate resistant transgenic maize, including steps of: introducing the DNA molecule according to claim 1 into a target maize to obtain a transgenic maize expressing the DNA molecule; the transgenic maize has an increased resistance to glyphosate as compared to the target maize.

16. The method according to claim 15, wherein the DNA molecule is introduced into the target maize via a recombinant vector comprising the DNA molecule.

17. A transgenic maize obtained by breeding with the method according to claim 15.


SEQUENCE LISTING (18 sequences)

SEQ ID NO: 1 - DNA, 1335 bp, Artificial Sequence
  Description of Artificial Sequence Synthetic polynucleotide
atggcgtgtttgcctgatgattcgggtccgcatgtcggccactccacgccacctcgccttgaccaggagccttgtaccttgagttcgcagaaaaccgtgaccgttacaccgcccaacttccccctcactggcaaggtcgcgccccccggctccaaatccattaccaaccgtgcgctgttgctggcggcattggccaagggcaccagccgtttgagcggtgcgctcaaaagcgatgacacgcgccacatgtcggtcgccctgcggcagatgggcgtcaccatcgacgagccggacgacaccacctttgtggtcaccagccaaggctcgctgcaattgccggcccagccgttgttcctcggcaacgctggcaccgccatgcgctttctcacggctgccgtggccaccgtgcaaggcaccgtggtactggacggcgacgagtacatgcaaaaacgcccgattggcccgctgctggctaccctgggccagaacggcatccaggtcgacagccccaccggttgcccaccggtcaccgtgcacggcatgggcaaggtccaggccaagcgtttcgagattgatggtggtttgtccagccagtacgtatcggccctgctgatgctcgcggcgtgcggcgaagcgccgattgaagtggcgctgaccggcaaggatatcggtgcccgtggctacgtggacctgaccctcgactgcatgcgtgccttcggggcccaggtggacgccgtggacgacaccacctggcgcgtcgcccccaccggctataccgcccatgattacctgatcgaacccgatgcgtccgccgccacgtatttgtgggccgcagaagtgctgaccggtgggcgtatcgacatcggcgtagccgcgcaggacttcacccagcccgacgccaaggcccaggccgtgattgcgcagttcccgaacatgcaagccacggtggtaggctcgcaaatgcaggatgcgatcccgaccctggcggtgctcgccgcgttcaacaacaccccggtgcgtttcactgaactggcgaacctgcgcgtcaaggaatgtgaccgcgtgcaggcgctgcacgatggcctcaacgaaattcgcccgggcctggcgaccatcgagggcgatgacctgctggtcgccagcgacccggccctggcaggcaccgcctgcaccgcactgatcgacacccacgccgaccatcgcatcgccatgtgctttgccctggccgggcttaaagtctcgggcattcgcattcaagacccggactgcgtggccaagacctaccctgactactggaaagccctggccagcctgggcgttcacctaaacgactga

SEQ ID NO: 2 - DNA, 1338 bp, Artificial Sequence
  Description of Artificial Sequence Synthetic polynucleotide
atggcgtgcctccctgacgattctggtccgcatgtcggacactccactccacctcgcctggaccaggagccctgtaccttgtcgtcccagaagacggttacggtgacgccacccaacttcccgttgactgggaaggtcgctccacctggctccaagtctatcaccaaccgtgcactgttgctggctgcgttggccaaagggaccagtcggttgagcggtgcgctcaagtcggatgacactcgccacatgtcggtggctctgaggcagatgggtgtcacgatcgacgaaccggatgacacgaccttcgtggtcacgagccaaggcagcctccagctgccagcccaaccgttgttcctcggcaatgctggcaccgctatgcgcttcctcacggctgcggtggccaccgtccaaggcacggtcgttctggatggcgatgagtacatgcagaagcgtcccatcggacccttgctggcgacgcttggacagaacggcatccaagtggattctcccaccggttgcccacccgtcaccgtgcacggcatgggcaaggtccaagcgaagcggttcgagatcgacggtgggctctcctcccagtacgtctcagccctgctcatgctcgctgcgtgtggcgaggcaccgatcgaggttgcgctcacaggcaaggacatcggtgccagagggtacgtggacctgaccctcgactgcatgagagcctttggagcacaggttgacgcagtggacgacacgacctggagggtcgctcccacagggtacaccgctcatgactatctcatcgaacccgacgccagcgctgcgacgtacttgtgggcagccgaggtcctgaccggtggcaggattgatattggcgttgctgcgcaggacttcacccaaccggacgccaaggctcaagccgtgattgcgcagttccctaacatgcaagccaccgtggttggctcccagatgcaggacgcgatcccgaccctggctgtcctcgcagccttcaacaatactcccgtgcgcttcactgagcttgccaaccttagggtcaaggagtgcgatcgcgtgcaagccctgcacgatgggctcaacgagatcaggccaggcctcgccaccatcgagggagacgacctgctcgtggccagcgacccagcgcttgcagggacagcctgcacagccctgatcgacacccacgctgaccatcgcatcgcgatgtgctttgccctggctggccttaaggtctcaggcattcgcattcaggaccctgactgcgtggccaagacctaccctgactactggaaggctcttgccagccttggcgttcacctcaacgactgatga

SEQ ID NO: 3 - DNA, 1350 bp, Artificial Sequence
  Description of Artificial Sequence Synthetic polynucleotide
ggatccatggcgtgcctccctgacgattctggtccgcatgtcggacactccactccacctcgcctggaccaggagccctgtaccttgtcgtcccagaagacggttacggtgacgccacccaacttcccgttgactgggaaggtcgctccacctggctccaagtctatcaccaaccgtgcactgttgctggctgcgttggccaaagggaccagtcggttgagcggtgcgctcaagtcggatgacactcgccacatgtcggtggctctgaggcagatgggtgtcacgatcgacgaaccggatgacacgaccttcgtggtcacgagccaaggcagcctccagctgccagcccaaccgttgttcctcggcaatgctggcaccgctatgcgcttcctcacggctgcggtggccaccgtccaaggcacggtcgttctggatggcgatgagtacatgcagaagcgtcccatcggacccttgctggcgacgcttggacagaacggcatccaagtggattctcccaccggttgcccacccgtcaccgtgcacggcatgggcaaggtccaagcgaagcggttcgagatcgacggtgggctctcctcccagtacgtctcagccctgctcatgctcgctgcgtgtggcgaggcaccgatcgaggttgcgctcacaggcaaggacatcggtgccagagggtacgtggacctgaccctcgactgcatgagagcctttggagcacaggttgacgcagtggacgacacgacctggagggtcgctcccacagggtacaccgctcatgactatctcatcgaacccgacgccagcgctgcgacgtacttgtgggcagccgaggtcctgaccggtggcaggattgatattggcgttgctgcgcaggacttcacccaaccggacgccaaggctcaagccgtgattgcgcagttccctaacatgcaagccaccgtggttggctcccagatgcaggacgcgatcccgaccctggctgtcctcgcagccttcaacaatactcccgtgcgcttcactgagcttgccaaccttagggtcaaggagtgcgatcgcgtgcaagccctgcacgatgggctcaacgagatcaggccaggcctcgccaccatcgagggagacgacctgctcgtggccagcgacccagcgcttgcagggacagcctgcacagccctgatcgacacccacgctgaccatcgcatcgcgatgtgctttgccctggctggccttaaggtctcaggcattcgcattcaggaccctgactgcgtggccaagacctaccctgactactggaaggctcttgccagccttggcgttcacctcaacgactgatgaggtacc

SEQ ID NO: 4 - DNA, 535 bp, Artificial Sequence
  Description of Artificial Sequence Synthetic polynucleotide
ttggcatgcacatacaaatggacgaacggataaaccttttcacgcccttttaaatatccgttattctaataaacgctcttttctcttaggtttacccgccaatatatcctgtcaaacactgatagtttaaggtcccaattgcgcacgtgcccaagcttctgcagtctagaggatcctaccgagctcgaattccggcccggggggtcgacccaggtacattaaaaacgtccgcaatgtgttattaagttgtctaagcgtcaatttgtttacaccacaatatatcctgccaccagccagccaacagctccccgaccggcagctcggcacaaaatcaccactcgatacaggcagccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggttt

SEQ ID NO: 5 - DNA, 2010 bp, Artificial Sequence
  Description of Artificial Sequence Synthetic polynucleotide
gcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctaga

SEQ ID NO: 6 - DNA, 67 bp, Artificial Sequence
  Description of Artificial Sequence Synthetic oligonucleotide
tatttttacaacaattaccaacaacaacaaacaacaaacaacattacaattactatttacaataacc

SEQ ID NO: 7 - DNA, 141 bp, Artificial Sequence
  Description of Artificial Sequence Synthetic polynucleotide
atggcgcccaccgtgatgatggcctcgtcggccaccgccgtcgctccgttccaggggctcaagtccaccgccagcctccccgtcgcccgccgctcctccagaagcctcggcaacgtcagcaacggcggaaggatccggtgc

SEQ ID NO: 8 - DNA, 491 bp, Artificial Sequence
  Description of Artificial Sequence Synthetic polynucleotide
ggtaccgggccccccctcgaggctgagtaaggttaactttgagtattatggcattggaaaagccattgttctgcttgtaatttactgtgttctttcagttttgttttcggacatcaagttaacaaaaaaaaaaaaaaaaaaaaaaaaatttaacaaaaaaaaaaaaaaaaaaaaaaaatttaacaaaaaaaaaaaaaaaaaaaaaaaaatttaaagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgacgcgatagaaaacaaaatatagcgcgcaaactaggataaattatcgcgcgcggtgtcatctatgttactagatcgg

SEQ ID NO: 9 - protein, 444 aa, Artificial Sequence
  Description of Artificial Sequence Synthetic polypeptide
MACLPDDSGPHVGHSTPPRLDQEPCTLSSQKTVTVTPPNFPLTGKVAPPGSKSITNRALLLAALAKGTSRLSGALKSDDTRHMSVALRQMGVTIDEPDDTTFVVTSQGSLQLPAQPLFLGNAGTAMRFLTAAVATVQGTVVLDGDEYMQKRPIGPLLATLGQNGIQVDSPTGCPPVTVHGMGKVQAKRFEIDGGLSSQYVSALLMLAACGEAPIEVALTGKDIGARGYVDLTLDCMRAFGAQVDAVDDTTWRVAPTGYTAHDYLIEPDASAATYLWAAEVLTGGRIDIGVAAQDFTQPDAKAQAVIAQFPNMQATVVGSQMQDAIPTLAVLAAFNNTPVRFTELANLRVKECDRVQALHDGLNEIRPGLATIEGDDLLVASDPALAGTACTALIDTHADHRIAMCFALAGLKVSGIRIQDPDCVAKTYPDYWKALASLGVHLND

SEQ ID NO: 10 - DNA, 4058 bp, Artificial Sequence
  Description of Artificial Sequence Synthetic polynucleotide
gcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccccgggtatttttacaacaattaccaacaacaacaaacaacaaacaacattacaattactatttacaataaccatggcgcccaccgtgatgatggcctcgtcggccaccgccgtcgctccgttccaggggctcaagtccaccgccagcctccccgtcgcccgccgctcctccagaagcctcggcaacgtcagcaacggcggaaggatccggtgcatggcgtgcctccctgacgattctggtccgcatgtcggacactccactccacctcgcctggaccaggagccctgtaccttgtcgtcccagaagacggttacggtgacgccacccaacttcccgttgactgggaaggtcgctccacctggctccaagtctatcaccaaccgtgcactgttgctggctgcgttggccaaagggaccagtcggttgagcggtgcgctcaagtcggatgacactcgccacatgtcggtggctctgaggcagatgggtgtcacgatcgacgaaccggatgacacgaccttcgtggtcacgagccaaggcagcctccagctgccagcccaaccgttgttcctcggcaatgctggcaccgctatgcgcttcctcacggctgcggtggccaccgtccaaggcacggtcgttctggatggcgatgagtacatgcagaagcgtcccatcggacccttgctggcgacgcttggacagaacggcatccaagtggattctcccaccggttgcccacccgtcaccgtgcacggcatgggcaaggtccaagcgaagcggttcgagatcgacggtgggctctcctcccagtacgtctcagccctgctcatgctcgctgcgtgtggcgaggcaccgatcgaggttgcgctcacaggcaaggacatcggtgccagagggtacgtggacctgaccctcgactgcatgagagcctttggagcacaggttgacgcagtggacgacacgacctggagggtcgctcccacagggtacaccgctcatgactatctcatcgaacccgacgccagcgctgcgacgtacttgtgggcagccgaggtcctgaccggtggcaggattgatattggcgttgctgcgcaggacttcacccaaccggacgccaaggctcaagccgtgattgcgcagttccctaacatgcaagccaccgtggttggctcccagatgcaggacgcgatcccgaccctggctgtcctcgcagccttcaacaatactcccgtgcgcttcactgagcttgccaaccttagggtcaaggagtgcgatcgcgtgcaagccctgcacgatgggctcaacgagatcaggccaggcctcgccaccatcgagggagacgacctgctcgtggccagcgacccagcgcttgcagggacagcctgcacagccctgatcgacacccacgctgaccatcgcatcgcgatgtgctttgccctggctggccttaaggtctcaggcattcgcattcaggaccctgactgcgtggccaagacctaccctgactactggaaggctcttgccagccttggcgttcacctcaacgactgatgaggtaccgggccccccctcgaggctgagtaaggttaactttgagtattatggcattggaaaagccattgttctgcttgtaatttactgtgttctttcagttttgttttcggacatcaagttaacaaaaaaaaaaaaaaaaaaaaaaaaatttaacaaaaaaaaaaaaaaaaaaaaaaaatttaacaaaaaaaaaaaaaaaaaaaaaaaaatttaaagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgacgcgatagaaaacaaaatatagcgcgcaaactaggataaattatcgcgcgcggtgtcatctatgttactagatcgg

SEQ ID NO: 11 - DNA, 10488 bp, Artificial Sequence
  Description of Artificial Sequence Synthetic polynucleotide
cacatacaaatggacgaacggataaaccttttcacgcccttttaaatatccgttattctaataaacgctcttttctcttaggtttacccgccaatatatcctgtcaaacactgatagtttaaggtcccaattgcgcacgtgcccaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccccgggtatttttacaacaattaccaacaacaacaaacaacaaacaacattacaattactatttacaataaccatggcgcccaccgtgatgatggcctcgtcggccaccgccgtcgctccgttccaggggctcaagtccaccgccagcctccccgtcgcccgccgctcctccagaagcctcggcaacgtcagcaacggcggaaggatccggtgcatggcgtgcctccctgacgattctggtccgcatgtcggacactccactccacctcgcctggaccaggagccctgtaccttgtcgtcccagaagacggttacggtgacgccacccaacttcccgttgactgggaaggtcgctccacctggctccaagtctatcaccaaccgtgcactgttgctggctgcgttggccaaagggaccagtcggttgagcggtgcgctcaagtcggatgacactcgccacatgtcggtggctctgaggcagatgggtgtcacgatcgacgaaccggatgacacgaccttcgtggtcacgagccaaggcagcctccagctgccagcccaaccgttgttcctcggcaatgctggcaccgctatgcgcttcctcacggctgcggtggccaccgtccaaggcacggtcgttctggatggcgatgagtacatgcagaagcgtcccatcggacccttgctggcgacgcttggacagaacggcatccaagtggattctcccaccggttgcccacccgtcaccgtgcacggcatgggcaaggtccaagcgaagcggttcgagatcgacggtgggctctcctcccagtacgtctcagccctgctcatgctcgctgcgtgtggcgaggcaccgatcgaggttgcgctcacaggcaaggacatcggtgccagagggtacgtggacctgaccctcgactgcatgagagcctttggagcacaggttgacgcagtggacgacacgacctggagggtcgctcccacagggtacaccgctcatgactatctcatcgaacccgacgccagcgctgcgacgtacttgtgggcagccgaggtcctgaccggtggcaggattgatattggcgttgctgcgcaggacttcacccaaccggacgccaaggctcaagccgtgattgcgcagttccctaacatgcaagccaccgtggttggctcccagatgcaggacgcgatcccgaccctggctgtcctcgcagccttcaacaatactcccgtgcgcttcactgagcttgccaaccttagggtcaaggagtgcgatcgcgtgcaagccctgcacgatgggctcaacgagatcaggccaggcctcgccaccatcgagggagacgacctgctcgtggccagcgacccagcgcttgcagggacagcctgcacagccctgatcgacacccacgctgaccatcgcatcgcgatgtgctttgccctggctggccttaaggtctcaggcattcgcattcaggaccctgactgcgtggccaagacctaccctgactactggaaggctcttgccagccttggcgttcacctcaacgactgatgaggtaccgggccccccctcgaggctgagtaaggttaactttgagtattatggcattggaaaagccattgttctgcttgtaatttactgtgttctttcagttttgttttcggacatcaagttaacaaaaaaaaaaaaaaaaaaaaaaaaatttaacaaaaaaaaaaaaaaaaaaaaaaaatttaacaaaaaaaaaaaaaaaaaaaaaaaaatttaaagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgacgcgatagaaaacaaaatatagcgcgcaaactaggataaattatcgcgcgcggtgtcatctatgttactagatcgggaattccggcccggggggtcgacccaggtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatg

SEQ ID NO: 12 - RNA, 14 nt, Unknown
  Description of Unknown Plant virus capsid protein gene
uauuuuuacaacaa

SEQ ID NO: 13 - DNA, 23 bp, Artificial Sequence
  Description of Artificial Sequence Synthetic primer
ggatcctatttttacaacaatta

SEQ ID NO: 14 - DNA, 22 bp, Artificial Sequence
  Description of Artificial Sequence Synthetic primer
ggtaccttccgccgttgctgac

SEQ ID NO: 15 - DNA, 21 bp, Artificial Sequence
  Description of Artificial Sequence Synthetic primer
cggctccaaatccattaccaa

SEQ ID NO: 16 - DNA, 20 bp, Artificial Sequence
  Description of Artificial Sequence Synthetic primer
gccacttcaatcggcgcttc

SEQ ID NO: 17 - DNA, 22 bp, Artificial Sequence
  Description of Artificial Sequence Synthetic primer
ccacctggctccaagtctatca

SEQ ID NO: 18 - DNA, 20 bp, Artificial Sequence
  Description of Artificial Sequence Synthetic primer
gcgtcaacctgtgctccaaa